(19) United States
(12) Patent Application Publication
(10) Pub. No.: US 2002/0013616 A1
(43) Pub. Date: Jan. 31, 2002

(54) Multilayer stents having enhanced flexibility and hoop strength

(76) Inventors: Andrew Carter, Saratoga, CA (US); Hsin-Yi Deng, Mountain View, CA (US); Ryan Kendall Pierce, Mountain View, CA (US)

Correspondence Address: Bret E. Field, Bozicevic, Field and Francis LLP, Suite 200, 200 Middlefield Road, Menlo Park, CA, 94025, US

(21) Appl. No.: 09/848,962

(22) Filed: May 4, 2001

Related U.S. Application Data
(60) Provisional application No. 60/202,276, filed on May 5, 2000.

(51) Int. Cl. A61F 2/06

(52) U.S. Cl. 623/1.15

(57) ABSTRACT

Multilayer stents, as well as methods for their use and kits comprising the same, are provided. The subject multilayer stents include at least two distinct layers or structures concentrically arranged in a manner sufficient to provide a multilayer stent that exhibits enhanced flexibility in a compressed state and enhanced hoop strength in an expanded state, as compared to a single layer stent of the same thickness. The subject multilayer stents find use in a variety of different applications, including vascular applications in which the stents are implanted into the vascular system of a patient.

### FIELD OF THE INVENTION

The field of this invention is stents.

### BACKGROUND OF THE INVENTION

The term “stent” is generically used in this application to describe structural devices that support living tissues. Stents are implanted in a body lumen for treating abnormal conditions. For example, stents have found use in maintaining the patency of collapsing and partially occluded blood vessels, particularly to prevent acute closure and restenosis after a vessel has been enlarged by angioplasty. Stents have also been used to reinforce other body lumens, such as the urinary tract, the bile tract, the intestinal tract, and the tracheobronchial tree.

Conventional stents are cut from a tube or formed from a wire that has been bent back and forth in a zig-zag pattern and wound in a circumferential direction to form one or more loops of a pre-determined circumference. Typically, the stent is radially expandable from a collapsed condition. It is desirable to minimize the diameter of the collapsed stent so that it can be delivered as unobtrusively as possible through the vasculature. Once in position it is expanded to the predetermined size, to support and reinforce the lumen.

The stent is normally inserted in the collapsed condition by a catheter during intraluminal delivery to the repair site. Once properly located, the stent is removed from the catheter and radially expanded until its circumference firmly contacts the interior wall of the lumen. Usually the radial expansion is caused by the dilation of an angioplasty balloon placed axially within the stent. Alternatively, the stent may be made from a shape memory metal, whereby the stent will automatically assume its expanded circumference as its temperature increases upon implantation, or stents can be made that expand through spring action.

An important attribute of the stent is its ability to provide radial support. This capability is a concern not only where the stent is being used to maintain the patency of the lumen in which it is located, but also where the stent is being used in conjunction with a prosthetic graft to keep the graft open and to hold it at the location at which it is implanted.

The patent literature contains descriptions of many different stent designs. A few of the more recent patents include U.S. Pat. No. 5,702,419, “Expandable, Intraluminal Stent”; U.S. Pat. No. 5,707,388, “High Hoop Strength Intraluminal Stent”; U.S. Pat. No. 5,707,387, Flexible Stent”; and U.S. Pat. No. 5,681,345, “Sleeve Carrying Stent”; Palmaz, U.S. Pat. No. 5,102,417, “Expandable intraluminal graft, and method and apparatus for implanting an expandable intraluminal graft”; and Sigwart, U.S. Pat. No. 5,443,500, “Intravascular stent”.

Scientific reviews of stent design and function may be found in Wong et al. (1996) Catheterization and Cardiovascular Diagnosis 39:413-419; Sniderman (1996) Progress in Cardiovascular Diseases, vol. XXXIX:141-164. Fontaine and dos Passos (1997) Journal of Vascular and Interventional Radiology 8:107-111 present an example of pre-clinical analysis for a prototype stent. Hong et al. (1997) Coronary Artery Disease 8:45-48, describe pre-clinical use of a self-expanding nitinol stent.

Features desirable in a stent are reviewed by Palmaz (1992) Cardiovasc. Intervent. Radiol. 15:279-284. A highly desirable stent would combine high lengthwise flexibility in the compressed state with high hoop strength in the expanded state. The present invention provides this and other useful features.

Relevant Literature

U.S. patents of interest include: U.S. Pat. Nos. 5,618,299; 5,645,559; 5,741,293; 5,723,003; 5,879,370; 5,935,162; 5,957,974; 5,964,798; 5,968,091; 5,980,565; 6,027,529. Also of interest are: WO 99/55257; EP 0 878 173 A1 and EP 0 536 164 B1.

### SUMMARY OF THE INVENTION

Multilayer stents, as well as methods for their use and kits comprising the same, are provided. The subject multilayer stents include at least two distinct layers or structures concentrically arranged in a manner sufficient to provide a multilayer stent that exhibits enhanced flexibility in a compressed state and enhanced hoop strength in an expanded state, as compared to a single layer stent of the same thickness. The subject multilayer stents find use in a variety of different applications, including vascular applications in which the stents are implanted into the vascular system of a patient.

### BRIEF DESCRIPTION OF THE FIGURES

FIGS. 1A and 1B show a stent according to one embodiment of the subject invention in a first compressed and second expanded state, respectively.

FIGS. 2A and 2B provide a two dimensional view of a layer of the dual stent shown in FIGS. 1A and 1B (which is a stent according to one of embodiment of the subject invention) in a compressed and expanded form, respectively.

### DESCRIPTION OF THE SPECIFIC EMBODIMENTS

Multilayer stents, as well as methods for their use and kits comprising the same, are provided. The subject multilayer stents include at least two distinct layers or structures concentrically arranged in a manner sufficient to provide a multilayer stent that exhibits enhanced flexibility in a compressed state and enhanced hoop strength in an expanded state, as compared to a single layer stent of the same thickness. The subject multilayer stents find use in a variety of different applications, including vascular applications in which the stents are implanted into the vascular system of a patient.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

As summarized above, the subject invention provides expandable multilayer stents that are highly flexible in their first, compressed state and exhibit high hoop strength in their second expanded state. As the subject stents are multilayer stents, they are made up of a plurality of distinct, concentric layers. As the multilayer stents are made of a plurality of concentric layers, the concentric layers are arranged in such a manner that they share a common axis, i.e. so that they are coaxial. The number of different or distinct layers in the multilayer stents may vary, where the number of distinct layers generally ranges from about 2 to 6, usually from about 2 to 4 and more usually from about 2 to 3, wherein in many embodiments the number of different layers in the subject stents is 2, such that the stents are dual layer stents made up of two concentric or coaxial layers.

The subject stents can exist in either a first, compressed state or a second expanded state, i.e. they are capable of going from a first compressed state to a second or expanded state. The first or compressed state is characterized by having a cross-sectional diameter that is smaller than the cross-sectional diameter of the expanded state, where the magnitude of this difference generally ranges from about 1 to 30 mm, usually from about 1 to 5 mm. The cross-sectional outer diameter of the compressed state typically ranges from about 1 to 2 mm, usually from about 1 to 1.5 mm, while the cross-sectional outer diameter in the expanded state typically ranges from about 2 to 30 mm, usually from about 2.5 to 6 mm. The longitudinal length of the stent may vary depending on the particular use for which the stent is developed, but typically ranges from about 8 to 60 mm, usually from about 8 to 30 mm and more usually from about 8 to 20 mm, where any difference in stent length between the compressed and expanded state generally does not exceed about 5%, usually does not exceed about 3-5% and more usually does not exceed about 3%.

The wall width W of the multilayer stent of the subject invention is the sum of the wall widths of each of the layers that make up the multilayer stent. For example, in a dual layer stent of the subject invention having a first inner layer with a wall width of X and a second outer layer with a wall width of Y, the wall width of the multilayer stent is X+Y=W. In the subject stents, W typically ranges from about 0.002″ to 0.02″, usually from about 0.003″ to 0.006″ and more usually from about 0.0035″ to 0.0045″.

A feature of the subject multilayer stents is that they exhibit high flexibility in the first, compressed state and high hoop strength in the second, expanded state. Flexibility of the subject stents is measured, evaluated, described or characterized in terms of the amount of force required to produce axial deformation in the stent, i.e. to bend the stent. The flexibility of the subject stents in the compressed state is greater than the flexibility of a single layer stent having the same configuration (where representative configurations are described in greater detail below) and a wall thickness W that is the same as that of the multilayer stent. The amount of enhancement in flexibility of the subject multilayer stent as compared to a corresponding single layer stent (i.e. one that has the same configuration and wall thickness W) is at least about 1.1 fold, usually at least about 1.5 fold and more usually at least about 1.75 fold, where in certain embodiments it is at least about 2 fold, usually at least about 3 fold and more usually at least about 4 fold. In many embodiments, the subject stents exhibit a flexibility in the first compressed state that is less than about 0.05, usually less than about 0.04 and more usually less than about 0.03 kgf/mm.

A second feature of the subject multilayer stents is that they exhibit high hoop strength in the expanded state. By hoop strength is meant the radial strength or compressive resistance of the multilayer stent in the expanded state. Compared with single layer stents of the same wall width W, the subject multilayer stents exhibit enhanced hoop strength, where the magnitude of the enhancement is typically at least about 1.1 fold, usually at least about 1.5 fold and more usually at least about 1.75 fold, where in certain embodiments it is at least about 2 fold, usually at least about 3 fold and more usually at least about 4 fold. Where hoop strength is measured in vivo by evaluating the % stent recoil (i.e. (balloon inflated diameter-stent diameter/balloon inflated diameter)×100), the in vivo acute recoil observed in the subject stents is less than about 5%, usually less than about 2% and in many embodiments less than about 1%.

Looking now at the individual stent layers in the subject multilayer stents, the individual wall layers of any given stent are generally of the same configuration, by which is meant that they have the same overall structure. By structure is meant the design of the stent. A number of different stent structures are known in the art, where such structures include tubular, mesh, graft, coil, etc. In principle, any convenient structure may be employed. In many embodiments, tubular structures are preferred, e.g. the tubular embodiment described in more detail infra.

The wall width of any given structure or layer in the multilayer stent, i.e. the difference between the inner and outer diameter of each structure, may be the same as or different from the wall width of any other structure in the multilayer structure. Where the wall widths of any two given structures in the stent differ, the magnitude of the difference generally does not exceed about 100%, usually about 50% and more usually about 25%. The wall width of any given structure typically ranges from about 0.001″ to 0.01″, usually from about 0.0015″ to 0.003″ and more usually from about 0.0015″ to 0.002″.

In the subject stents, the different layers that make up the stents are arranged in a such a manner so as to provide for the enhanced flexibility and hoop strength characteristics, as described supra. Generally, one layer in the subject stents is aligned relative to another layer so as to provide the requisite flexibility and hoop strength characteristics. In many embodiments, this alignment requires that one layer be offset from the other layer by an amount that provides for these desired physical characteristics, where the particular amount of offset that is required depends on the particular configurations of the individual layers.

In many embodiments, the subject stents are further characterized in that the stents provide for high surface coverage of the arterial wall or other structure of the body in the expanded state. By high surface coverage is meant that a substantially large portion of the arterial wall or other structure of the body is covered by a stent element when the stent is in the expanded state. By substantially large proportion is meant at least about 15, usually at least about 25 and more usually at least about 25 to 40%.

Where desired, the different layers of the stent may be attached to each other at one or more sites in order to provide for the desired alignment of the disparate layers in the expanded state. When present, the number of sites of attachment or connecting points is kept to a minim so as to provide for the desired flexibility, at least in the compressed state. As such, the number of different connecting points, e.g. welds, is generally at least 1, usually at least 2 and more usually at least 3. In principle, a large number of different connecting points may be present. However, in many embodiments, the number of connecting points does not exceed about 100, usually does not exceed about 50 and more usually does not exceed about 10. The connecting points may be arranged in any convenient manner, e.g. at one or both ends of the stent, along a longitudinal line of the stent, helical or spiral line of the stent, etc. The connecting points may be secured in any convenient manner, e.g. by welding, with adhesive, etc.

Where desired, the stent may be covered with a “sock” or graft of flexible material, as known in the art. The sock may be completely on the inside of the stent; completely outside the stent; or woven in between the elements of the stent, depending on the particular stent embodiment. Conveniently, the sock is attached with stitches or glue. The sock forms a synthetic vessel, where the vessel is a tubular member usually having a substantially uniform bore. Suitable materials for the vessel include, for example, expanded polytetrafluoroethylene (e-PTFE) and dacron. High porosity ePTFE may be used for some purposes, where the slit-like fissures in the vessel well are in the range of 90 μm in size. For vascular repair, the vessel will generally be at least about 1 mm in internal diameter, more usually at least about 15 to 25 mm in diameter, and not more than about 50 mm in diameter.

To reduce the thrombogenicity of the graft, the vessel may be sodded or seeded with endothelial cells. Sodding procedures place the cells directly onto the polymeric internal surface of the vessel as well as into the interstices of the vessel, generally under mild pressure. For example, one termini of the vessel may be clamped, and the cells injected with a syringe through the open end. The vessel is porous to water, and so the media is forced through the interstices of the wall, while the cells are retained.

Seeding procedures mix the cells with blood or plasma, and then add to the vessel during the pre-clotting period. There are several versions of the technique known as seeding. The synthetic grafts can be coated with collagen or fibronectin prior to the addition of endothelial cells into the lumen. The synthetic graft is then incubated in vitro with rotation to allow the binding of the endothelial cells to the luminal surface. After several hours or days culture, the graft can be implanted. Alternatively, autologous blood can be forced under pressure through the interstices of the synthetic graft to allow retention of blood cells and protein onto and into the graft prior to addition of the endothelial cells (either passively are actively under pressure). A third alternative is to mix the endothelial cells with the blood prior to the application onto and into the graft.

Endothelial cells may be genetically modified to express factors that encourage the growth of endothelial cells, e.g. VEGF; PlGF; TGF-β1; aFGF and bFGF; and hepatocyte growth factor; or a protein that inhibits the growth of intimal cells, for example, inducible nitric oxide synthase (iNOS) or endothelial cell nitric oxide synthase (ecNOS). Proteins that inhibit thrombosis, e.g. tissue plasminogen activator (tPA), urokinase, and streptokinase, are also of interest.

In certain embodiments, the stent may include a reservoir of biologically active materials, e.g. antibiotics, anti-thrombogenic factors, growth factors, etc. Such a reservoir may be a coating on the stent or elements thereof, embedded in plastics or the graft, deposited as a gel inside the spring coils, etc. Often stent grafts are impregnated with biocompatible substances or are coated with heparin or hydrogel.

In certain embodiments, active agents are incorporated into or otherwise associated with the stents, where active agents include drugs, radiation emitting agents, e.g., radioactive elements such as coatings, or other biological factors, such as those described above, which agents may be incorporated into one or more stent layers, either as surface coatings or contained within pores or holes in the stent structure. In certain embodiments, one may incorporated such active agents into the inner stent layer or layers, thereby shielding the vascular wall from direct contact with the treated stent and diminishing edge effects. The extra surface area provided by the multiple layers of the subject stents, as compared to a single-layer stent, can be exploited to increase the amount of active agent, e.g., drug, the device can contain, compared to single-layer, drug-coated stents.

Specific drug or therapeutic agents of interest include, but are not limited to: therapeutic and pharmaceutic agents such as, but not limited to: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which don't have the capacity to synthesize their own asparagine); antiproliferative/antimitotic alkylating agents such as nitrogen mustards(mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin),trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (i.e. estrogen); Anticoaglants (heparin, synthetic heparin salts and other inhibitors of thrombin); fibrinolytic agents (such as tissue plasminogen activator, streptokinase and urokinase); antiplatelet: (aspirin, dipyridamole, ticlopidine, clopidogrel, abciximab); antimigratory; antisecretory (breveldin); antiinflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; Indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressive: (cyclosporine, tacrolimus (FK-506), sirolimus (rapamycin), azathioprine, mycophenolate mofetil); Angiogenic: vascular endothelial growth factor (VEGF), fibroblast growth factor (FGF); nitric oxide donors; anti-sense olgio nucleotides and combinations thereof. Radiation can be provided by employing Sm-153, Dy-165, Ho-166, Er-169, P-32, Y-90, I-131, Re-186, Re-188, Pd-109 or Au-198 as beta-ray emitting nuclides and Ir-192, Co-57, Co-60, V-48 or I-125 as gamma-ray emitting nuclides and Pd-103 as both gamma- and beta-ray emitting nuclides.

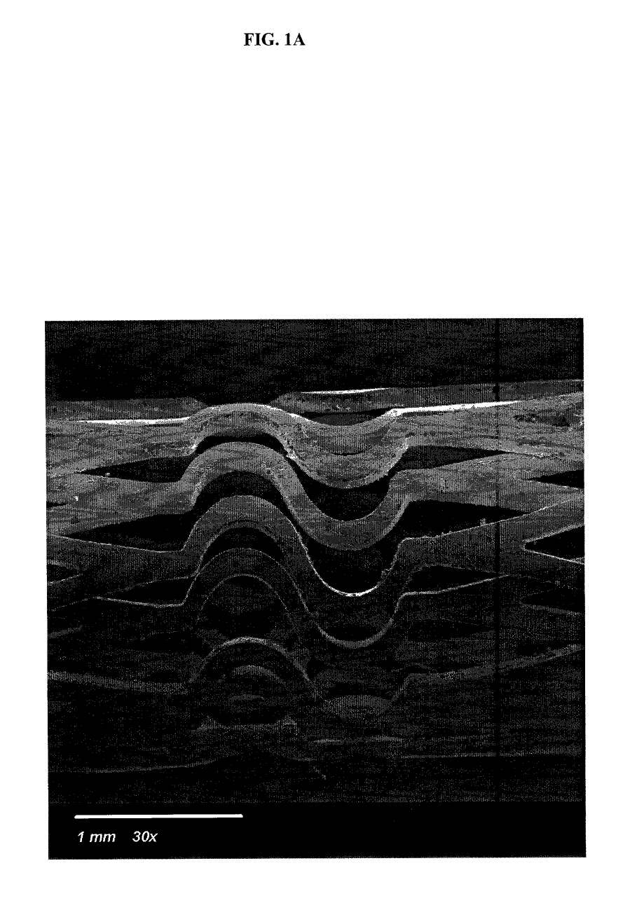

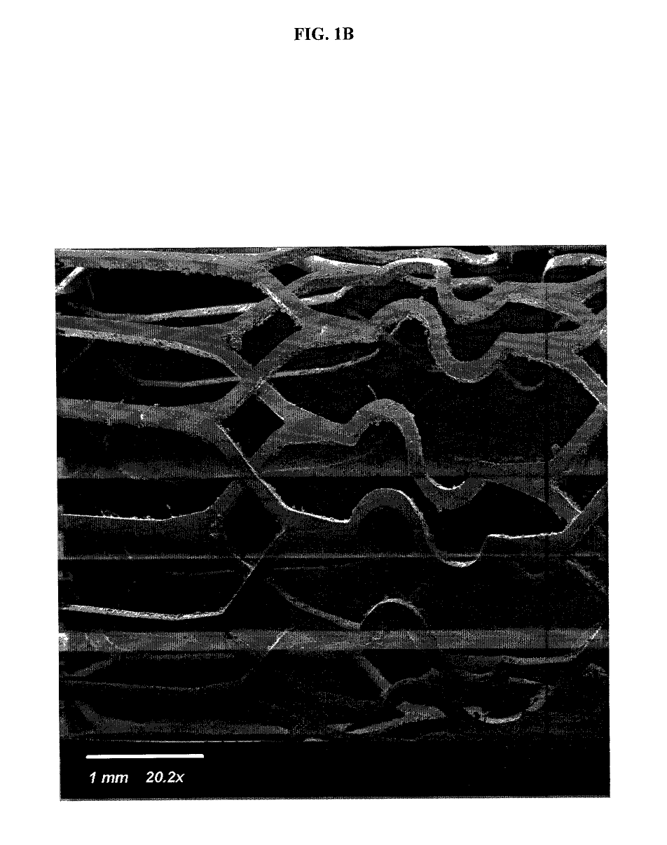

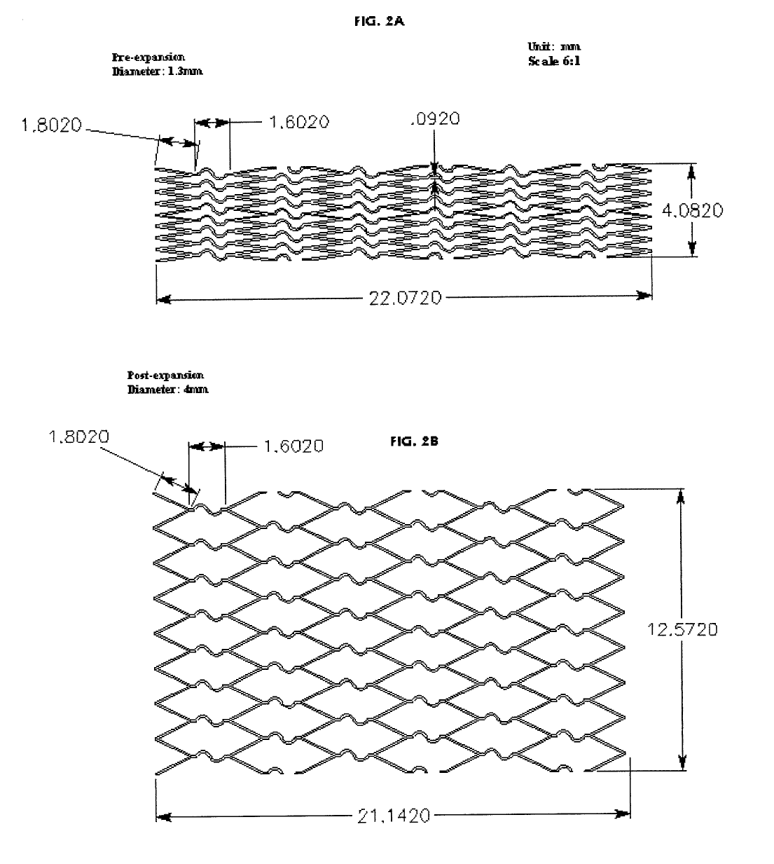

The subject stents having been described in general terms, a representative embodiment of the subject multilayer stents is now described in greater detail in terms of the figures. FIGS. 1A and 1B provide a picture of a dual layer stent of the subject invention in the compressed and expanded state, respectively. FIGS. 2A and 2B provide a two dimensional representation of one of the layers of the stent shown if FIGS. 1A and 1B. As can be seen in the figures, each layer of the stent is made up of stacked hexagonal elements joined to each other by longitudinal curvilinear elements. Specifically, each layer is made up of hexagonal elements joined at their top and bottom to additional hexagonal units and at their sides to longitudinal elements. The area of any given hexagonal element typically ranges from about 0.5 to 100 mm², usually from about 1 to 8 mm , and the length of any hexagonal side ranges from about 1 to 5 mm, usually from about 1 to 3 mm. As can be seen, the longitudinal curvilinear elements have one inflection point located at substantially their midpoint. The length of the curvilinear longitudinal elements typically ranges from about 1 to 8 mm, usually from about 1 to 4 mm. In many embodiments, the width of the longitudinal elements is less than the width of the hexagonal elements, where this magnitude of the difference in widths typically ranges from about 0 to 50%, usually from about 10 to 40% and more usually from about 15 to 25%. As such, the width of any given longitudinal element typically ranges from about 0.002″ to 0.01″, usually from about 0.003″ to 0.006″ and more usually from about 0.0035″ to 0.0045″ while the width of any given hexagonal element (i.e. side in one of the hexagons) typically ranges from about 0.0025″ to 0.015″, usually from about 0.003″ to 0.004″.

In this embodiment, the wall width of each of the tubular structures is substantially the same, ranging from about 0.0025″ to 0.025″, usually from about 0.003″ to 0.015″ and more usually from about 0.003″ to 0.006″. The inner diameter of the outer structure is only slightly larger than the outer diameter of the inner structure in the compressed state, where the difference in these diameters is merely sufficient to place the inner structure inside the outer structure, and generally ranges from about 0.01 to 0.5 mm, usually from about 0.01 to 0.25 mm and more usually from about 0.01 to 0.03 mm.

In this embodiment, the two tubular layers are offset from each other in a manner sufficient to provide for the requisite high flexibility, hoop strength and vessel wall coverage (i.e. through reduced cell size). The amount of offset may vary, but typically ranges from about 0.33 to 0.66 of the circumferential distance between any two adjacent longitudinal members in a layer of the stent, and in many embodiments is about or is 0.50 of the circumferential distance between any two adjacent longitudinal members in a layer of the stent. In many embodiments of this representative stent structure, at least one of the contact points between the two stent layers is welded, e.g. laser welded, or stably contacted/attached using some analogous means. The number of welds or analogous stable contacts/attachments is sufficient to ensure the stability of the desired offset geometry and yet provide for the requisite flexibility in the compressed state. Typically, the number of welds ranges from about 1 to 10, usually from about 1 to 6 and more usually from about 2 to 4.

The stents of the subject invention may be fabricated from any convenient material(s). Of particular interest are biologically compatible materials. Biologically compatible metals include stainless steel, titanium, tantalum, gold, platinum, copper and the like, as well as alloys of these metals. Low shape memory plastic may also be used. Alternatively the filament is formed from a shape-memory plastic or alloy, such as nitinol, which automatically transforms from one shape to another as its temperature passes through a critical point.

The subject stents may be fabricated using any convenient protocol. A representative protocol for the fabrication of a stent according to the subject invention, i.e. the stent shown in FIGS. 1A & B, is provided in the experimental section, supra.

The multilayer stents of the subject invention find use in a variety of different applications. Stents are commonly used to open blood vessels, e.g. clearing obstructions, and to repair damage to vascular tissues, e.g. arteries and veins. The stents are used conventionally, for preventing restenosis or other narrowing of vessels, to provide support for the vessel at the site of an aneurysm or other weakening of the vessel wall. The use of stents for the support of blood vessels is well known in the art and need not be further elaborated here. A modification of stents where there is a flexible cover attached to the stent frame is commonly referred to as stent graft. The purpose of stent grafts is to seal off vascular abnormalities, such as aneurisms.

In addition to blood vessels, other vessels of the body may be repaired with a stent, including the trachea for breathing disorders, renal and urethral tubules, fallopian tubes for the treatment of infertility, eustachian tubes for the treatment of chronic ear infection and other hearing disorders, large and small intestines, etc. The stent design is not limited to any particular body tissue, but will be manufactured with a size, expansion, and radial stiffness suitable for the different purposes.

The recipient for the stent may be any mammalian species, including canines; felines; equines; bovines; ovines; etc. and primates, particularly humans. Animal models, particularly small mammals, e.g. murine, lagomorpha, etc. are of interest for experimental investigations.

The stents are useful for any vascular surgery, such as may be used in any situation in which the flow of blood through a vessel has been compromised. There are a variety of conditions where there is restricted blood flow through that vessel. Occlusive vascular conditions of interest include atherosclerosis, graft coronary vascular disease after transplantation, vein graft stenosis, peri-anastomatic prosthetic graft stenosis, restenosis after angioplasty, coronary artery disease, peripheral vascular disease or other forms of occlusive arterial disease, and the like.

Any convenient method for the placement of the stent may be used. As known in the art, a stent is inserted into a catheter for delivery in a non-expanded condition. The catheter is used to thread the stent through the vasculature, to the site for placement. The stent is then pushed or otherwise maintained in position while the catheter is withdrawn. In some cases, it may be desirable for the stent to continue to expand after the insertion procedure is performed. A balloon catheter may be positioned inside the stent in situ after the original placement, and used to further expand the diameter. If the stent has a self-expanding design, then the stent will continue to self-expand in situ as much as the vessel will allow, or until it reaches the maximum diameter.

Also provided are kits that at least include the subject stents. The subject kits at least include a multilayer stent of the subject invention and instructions for how to use the stent in a procedure. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc. In addition, the subject kits may also include a catheter delivery means for use in delivering the catheter to the site of implantation, where the catheter may be a balloon catheter etc., depending on the particular design of the stent, e.g. whether it is self-expanding, and the like.

The following examples are offered by way of illustration and not by way of limitation.

### Experimental

I. Stent Fabrication

Prototypes of one embodiment of the device were made by laser-cutting the stent pattern shown in FIG. 2 from stainless steel cylinders with an approximate circumference of 4 mm. After laser-cutting, one structure was expanded minimally by inserting a metal rod; another structure was then inserted manually and rotated to attain a preferred offset geometry. Although laser-welding was not performed on these prototypes, it is expected that such an operation could be used to maintain the preferred offset.

II. Use of Stent

The multi-layer stent is implanted in peripheral or coronary arterial lesions using standard commercially available guidewires and guiding catheters with fluoroscopic and/or intravascular ultrasound guidance. In brief, the balloon expandable multi-layer stent is premounted or hand crimped on a standard balloon angioplasty catheter. After completion of coronary angiography and positioning of a guidewire distal to the arterial obstruction, the operator advances the stent and balloon catheter to the site of the lesion. Pre-treatment of the lesion with balloon angioplasty or atherectomy may be required for severe stenosis or heavily calcified lesions to allow stent delivery and deployment. Angiography is recommended to confirm correct position of the stent in the lesion before deployment. Stent deployment is accomplished with a single balloon inflation at 6 to 9 ATM pressure. Additional balloon inflations with higher inflation pressures or larger diameter balloons may be used to fully expand the stent in the lesion. Coronary angiography or intravascular ultrasound is used to document optimal stent deployment. Alternatively, a mechanical or shape memory alloy self-expanding multi-layer stent is delivered to the arterial lesion in a low profile sheath over the guidewire. The self-expanding multi-layer stent deployment occurs by withdrawal or removal of the constraining sheath to allow stent expansion.

It is evident from the above results and discussion that the present invention represents an important advance in the field of stents. Specifically, the stents of the subject invention exhibit enhanced flexibility in the compressed state and enhanced hoop strength in the expanded state as compared to corresponding single layer stents of the same wall thickness. In addition, the multilayer stent design of the subject stents provides for greater wall coverage, therefore reducing the risk of restenosis following implantation. Accordingly, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A multilayer stent, said stent comprising:
at least two layers having a combined wall thickness W, wherein said multilayer stent is capable of going from a first compressed state to a second expanded state and exhibits enhanced flexibility in said first compressed state and enhanced hoop strength in said second expanded state as compared to a single layer stent of wall thickness W.

2. The multilayer stent according to claim 1, wherein W ranges from about 0.02 to 3 mm.

3. The multilayer stent according to claim 1, wherein said first and second layers are arranged relative to one another in a manner sufficient to provide for said enhanced flexibility and hoop strength.

4. The multilayer stent according to claim 1, wherein said at least two layers have tubular, coil, mesh or graft configurations.

5. The multilayer stent according to claim 1, wherein said at least two layers have the same configuration.

6. The multilayer stent according to claim 1, wherein said stent comprises two layers.

7. The multilayer stent according to claim 1, wherein said at least two layers are attached to each other at at least one location.

8. A dual layer stent comprising:
a first tubular structure of wall thickness X; and
a second tubular structure of wall thickness Y;
wherein said first and second tubular structures are concentrically arranged so as to produce a dual layer stent capable of going from a first compressed state to a second expanded state, wherein said dual layer stent exhibits enhanced flexibility in said first compressed state and enhanced hoop strength in said second expanded state as compared to a single layer stent of wall thickness W, wherein X+Y=W.

9. The dual layer stent according to claim 8, wherein W ranges from about 0.02 to 3 mm.

10. The dual layer stent according to claim 8, wherein said first and second tubular structures are welded to each other at least one site.

11. The dual layer stent according to claim 8, wherein said first and second tubular structures have the same configuration.

12. A dual layer stent comprising:
first and second concentric tubular structures having the same the configuration and of identical wall thickness X ranging from about 0.002″ to 0.02″, wherein said concentric tubular structures are arranged so as to produce a dual layer stent capable of going from a first compressed state to a second expanded state, wherein said dual layer stent exhibits enhanced flexibility in said first compressed state and enhanced hoop strength in said second expanded state as compared to a single layer stent of wall thickness 2X.

13. The dual layer stent according to claim 12, wherein said dual layer stent exhibits a longitudinal flexibility in said first compressed state ranging from about 0.03 to 0.05 kgf/mm.

14. The dual layer stent according to claim 12, wherein said dual layer stent exhibits a hoop strength in said second expanded state ranging from about 0 to 5% recoil.

15. The dual layer stent according to claim 12, wherein said first and second tubular structures have a configuration substantially similar to or identical to the structures shown in FIG. 1.

16. In a method in which a stent is deployed in a physiological location, the improvement comprising:
employing a multilayer stent according to claim 1 in said procedure.

17. The method according to claim 16, wherein said physiological location is an intravascular site.

18. A kit comprising:
a stent according to claim 1; and
instructions for using said stent.

19. The kit according to claim 18, wherein said kit comprises a stent deployment means.

20. The kit according to claim 18, wherein said deployment means comprises a balloon catheter.
